(19) United States
(12) Patent Application Publication
(10) Pub. No.: US 2008/0243234 A1
(43) Pub. Date: Oct. 2, 2008

(54) Magnesium Alloy Stent

(75) Inventor: Josiah Wilcox, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/691,548

(22) Filed: Mar. 27, 2007

(51) Int. Cl. A61F 2/06

(52) U.S. Cl. 623/1.39; 977/910

(57) ABSTRACT

A method for treating a vascular condition includes delivering a magnesium alloy stent framework to a target region of a vessel, leaching at least a portion of magnesium from the magnesium alloy stent framework, and forming a plurality of pores within the stent framework of the stent based on the leaching.

### TECHNICAL FIELD

This invention relates generally to medical devices for treating vascular problems, and more particularly to a stent with a magnesium alloy.

### BACKGROUND OF THE INVENTION

Stents have become popular medical devices. One difficulty with such devices is obtaining a high degree of biocompatibility. Prior attempts to improve biocompatibility have focused on suppressing proliferation of vessel wall tissue around the stent framework.

It would be desirable, therefore, to overcome the limitations and disadvantages inherent in the devices described above.

### SUMMARY OF THE INVENTION

A first aspect of the invention provides a method for treating a vascular condition includes delivering a magnesium alloy stent framework to a target region of a vessel, leaching at least a portion of magnesium from the magnesium alloy stent framework, and forming a plurality of pores within the stent framework of the stent based on the leaching.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is an illustration of a system for treating a vascular condition including a magnesium alloy stent coupled to a catheter, in accordance with one embodiment of the current invention;

FIG. 2A is a cross-sectional perspective view of a magnesium alloy stent framework, in accordance with one embodiment of the current invention;

FIG. 2B is a cross-sectional perspective view of a magnesium alloy stent framework, in accordance with one embodiment of the current invention;

FIG. 2C is a cross-sectional perspective view of a magnesium alloy stent framework, in accordance with one embodiment of the current invention;

FIG. 2D is a cross-sectional perspective view of a magnesium alloy stent framework, in accordance with one embodiment of the current invention;

FIG. 3 is a flow diagram of a method of treating a vascular condition, in accordance with one embodiment of the current invention; and

FIG. 4 is a flow diagram of a method of treating a vascular condition, in accordance with one embodiment of the current invention.

### DETAILED DESCRIPTION

The invention will now be described by reference to the drawings wherein like numbers refer to like structures.

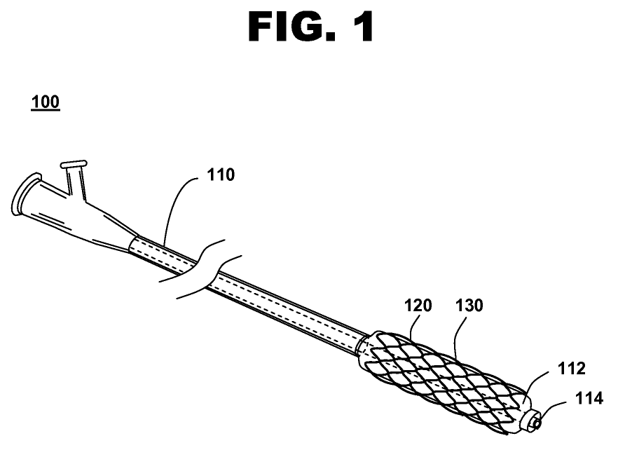

FIG. 1 shows an illustration of a system for treating a vascular condition, comprising a magnesium alloy stent coupled to a catheter, in accordance with one embodiment of the present invention at 100. Magnesium alloy stent with catheter 100 includes a magnesium alloy stent 120 coupled to a delivery catheter 110. Magnesium alloy stent 120 includes a stent framework 130. In one embodiment, at least one drug coating, or a drug-polymer layer, is applied to a surface of the stent framework.

Insertion of magnesium alloy stent 120 into a vessel in the body helps treat, for example, heart disease, various cardiovascular ailments, and other vascular conditions. Catheter-deployed magnesium alloy stent 120 typically is used to treat one or more blockages, occlusions, stenoses, or diseased regions in the coronary artery, femoral artery, peripheral arteries, and other arteries in the body. Treatment of vascular conditions may include the prevention or correction of various ailments and deficiencies associated with the cardiovascular system, the cerebrovascular system, urinogenital systems, biliary conduits, abdominal passageways and other biological vessels within the body.

The stent framework comprises an alloy comprising magnesium and other substances. In one embodiment, the alloy comprises magnesium and cobalt-chromium. In other embodiments, the magnesium is replaced with another sacrificial substance intended to leach into the body upon deployment.

Catheter 110 of an exemplary embodiment of the present invention includes a balloon 112 that expands and deploys the magnesium alloy stent within a vessel of the body. After positioning magnesium alloy stent 120 within the vessel with the assistance of a guide wire traversing through a guide wire lumen 114 inside catheter 110, balloon 112 is inflated by pressurizing a fluid such as a contrast fluid or saline solution that fills a tube inside catheter 110 and balloon 112. Magnesium alloy stent 120 is expanded until a desired diameter is reached, and then the contrast fluid is depressurized or pumped out, separating balloon 112 from magnesium alloy stent 120 and leaving the magnesium alloy stent 120 deployed in the vessel of the body. Alternately, catheter 110 may include a sheath that retracts to allow expansion of a self-expanding version of magnesium alloy stent 120.

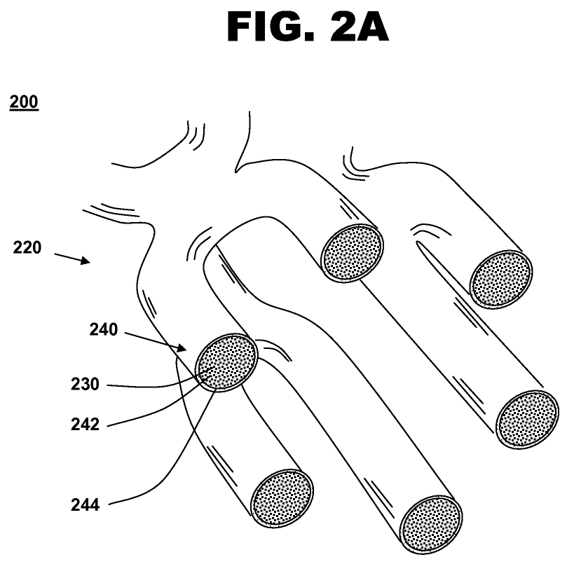

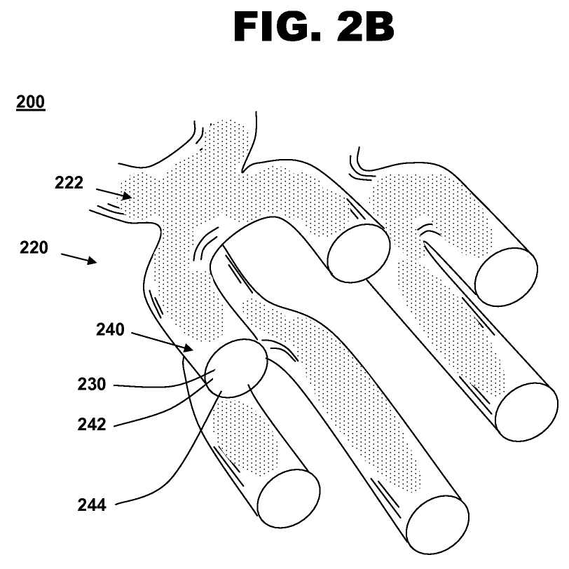

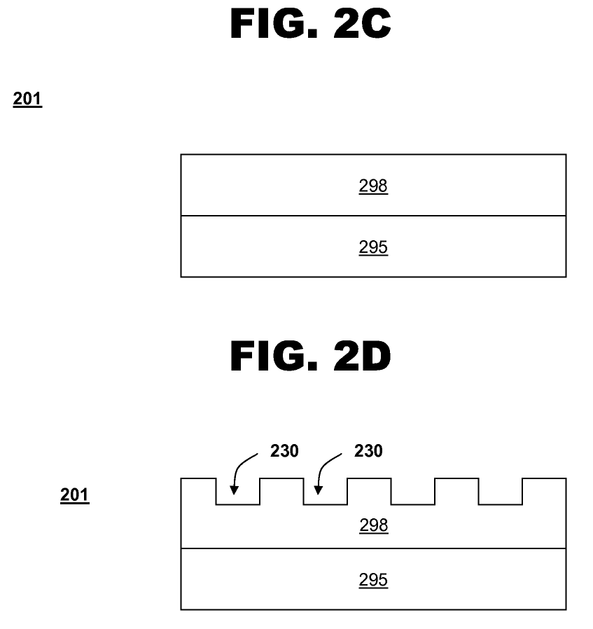

FIG. 2A shows a cross-sectional perspective view of a magnesium alloy stent, in accordance with one embodiment of the present invention at 200. A magnesium alloy stent 220 includes a stent framework 230. FIG. 2A illustrates the magnesium alloy stent prior to leaching of the magnesium from the stent framework.

Stent framework 230 comprises a metallic base formed of magnesium and other elements, such as cobalt-chromium, stainless steel, nitinol, tantalum, MP35N alloy, platinum, titanium, a chromium-based alloy, a suitable biocompatible alloy, a suitable biocompatible material, a biocompatible polymer, or a combination thereof. In one embodiment, the alloy does not include yttrium, neodymium, or zirconium. As the stent framework comes in contact with the blood stream and vessel wall tissue, the magnesium within the stent framework leaches out of the stent framework and into the body. As the magnesium leaches out of the stent framework, a pore or nanopore is left in the space previously occupied by the leached magnesium. In addition, the leached magnesium may reduce restenosis for at least some period of time. Tissue ingrowth into the pores may improve biocompatibility. The distribution of the formed pores can be controlled into a desired pattern in one embodiment. For example, the formed pores can assume a particular pattern, such as sinusoid, quincunx, or other. Alternatively, the formed pores can be dispersed on only a single side of the stent, such as the side of the stent opposite a lumen formed by the stent framework. In another embodiment, the distribution of the formed pores is uncontrolled.

It is important to note that the magnesium alloy forms the stent framework, and although the stent framework may be further coated, such as with drugs, or a magnesium layer, the term magnesium alloy stent framework means that the stent framework (such as stent struts) includes magnesium and not that a layer of magnesium is coated onto a stent framework.

In one embodiment, a drug coating 240 is disposed on stent framework 230. In certain embodiments, drug coating 240 includes at least one drug layer 242. In other embodiments, at least one coating layer 244 is disposed over the stent framework, and can envelop the drug coating layer. For example, drug layer 242 includes at least a first therapeutic agent. In one embodiment, coating layers 244 include magnesium. In one embodiment, the coating layers are sputter coats. In other embodiments, the magnesium coating is applied using another appropriate technique, such as vacuum deposition, dipping, or the like. In one embodiment, the coating layer is a topcoat.

Although illustrated with one set of drug layers and coating layers, multiple sets of drug and coating layers may be disposed on stent framework 230. For example, ten sets of layers, each layer on the order of 0.1 micrometers thick, can be alternately disposed on stent framework 230 to produce a two-micrometer thick coating. In another example, twenty sets of layers, each layer on the order of 0.5 micrometers thick, can be alternately disposed on stent framework 230 to produce a twenty-micrometer thick coating. The drug layers and the coating layers need not be the same thickness, and the thickness of each may be varied throughout drug coating 240. In one example, at least one drug layer 242 is applied to an outer surface of the stent framework. The drug layer can comprise a first therapeutic agent such as camptothecin, rapamycin, a rapamycin derivative, or a rapamycin analog. In another example, at least one coating layer 244 comprises a magnesium layer of a predetermined thickness. In one embodiment, the thickness of the magnesium coating is selected based on expected leaching rates, while in other embodiments, the thickness is selected based on the drug maintained in place between the magnesium alloy stent framework surface and the magnesium layer. In another embodiment, the thickness of the magnesium layer is variable over the length of the stent framework. Drug or magnesium elution refers to the transfer of a therapeutic agent from drug coating 240 to the surrounding area or bloodstream in a body. The amount of drug eluted is determined as the total amount of therapeutic agent excreted out of drug coating 240, typically measured in units of weight such as micrograms, or in weight per peripheral area of the stent.

FIG. 2B illustrates the stent 200 of FIG. 2A after leaching of the magnesium from the stent framework results in a plurality of pores 222 within the surface of the stent.

FIGS. 2A and 2B illustrate the stent framework as substantially tubular in cross-section. However, alternate geometric arrangements are contemplated. For example, FIG. 2C illustrates a stent framework cross-section using a single strut of the framework with a substantially planar construction. Magnesium alloy stent 201 includes a base portion 295 and magnesium alloy portion 298. Magnesium alloy portion 298 is opposite the lumen defined by the stent struts, while base portion 295 defines the outer diameter of the lumen. Stent 201 is manufactured by attaching a conventionally formed base stent surface 295 with a magnesium-alloyed portion 298. In one embodiment, such a construction results in formation of nanopores within the magnesium alloy portion 298, while reducing formation of nanopores in the base portion 295 on a side exposed to the bloodstream. Reduction in the formation of nanopores where the stent surface is exposed to the bloodstream may reduce cavitation within the blood flow and improve anti-thrombotic properties. FIG. 2D illustrates the stent strut 201 after the magnesium has leached from magnesium-alloyed portion 298, including a plurality of pores 299. Other geometric strut configurations are also anticipated, as well as variable configurations

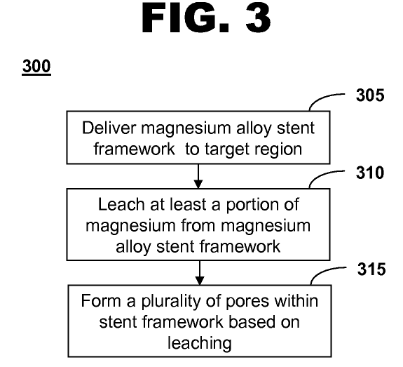

FIG. 3 shows a flow diagram of a method of treating a vascular condition, in accordance with one embodiment of the present invention at 300. Method 300 begins by delivering a magnesium alloy stent framework to a target region of a vessel at step 305.

When ready for delivery, the magnesium alloy stent with the magnesium alloy stent framework is inserted into a vessel of the body. The magnesium alloy stent is inserted typically in a controlled environment such as a catheter lab or hospital. A delivery catheter, which helps position the magnesium alloy stent framework in a vessel of the body, is typically inserted through a small incision of the leg and into the femoral artery, and directed through the vascular system to a desired place in the vessel. Guide wires threaded through an inner lumen of the delivery catheter assist in positioning and orienting the magnesium alloy stent framework. The position of the magnesium alloy stent and framework may be monitored, for example, with a fluoroscopic imaging system or an x-ray viewing system in conjunction with radiopaque markers on the magnesium alloy stent, radiopaque markers on the delivery catheter, or contrast fluid injected into an inner lumen of the delivery catheter and into an inflatable catheter balloon that is coupled to the magnesium alloy stent. The stent is deployed, for example, by expanding the stent framework with a balloon or by extracting a sheath that allows a self-expandable stent to enlarge after positioning the stent at a desired location within the body. Before clinical use, the stent is sterilized by using conventional medical means.

Once delivered, at least a portion of the magnesium within the magnesium alloy stent framework is leached out of the magnesium alloy stent framework, as seen at block 310. The magnesium leaches out over a period of time, and in certain embodiments, has a therapeutic effect.

As the magnesium leaches from the magnesium alloy stent framework, a plurality of pores is formed in the magnesium alloy stent framework based on the leaching, at block 315. These pores can be nanopores, dips, pits, channels, or other physical surface alteration.

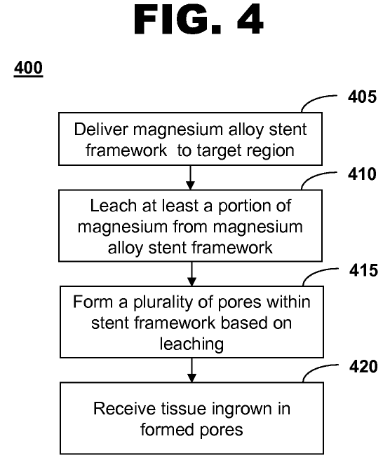

FIG. 4 shows a flow diagram of a method of treating a vascular condition, in accordance with one embodiment of the present invention at 400. Method 400 begins by delivering a magnesium alloy stent framework to a target region of a vessel at step 405. In one embodiment, step 405 is implemented in a similar fashion as step 305.

Once delivered, at least a portion of the magnesium within the magnesium alloy stent framework is leached out of the magnesium alloy stent framework, as seen at block 410. The magnesium leaches out over a period of time, and in certain embodiments, has a therapeutic effect.

As the magnesium leaches from the magnesium alloy stent framework, a plurality of pores is formed in the magnesium alloy stent framework based on the leaching, at block 415. These pores can be nanopores, dips, pits, channels, or other physical surface alteration. The formed pores receive at least some tissue ingrowth at step 420. The tissue ingrowth include tissue growth into the pores, as well as tissue growth around the stent framework.

In one embodiment, prior to deployment into a patient body, the magnesium alloy stent framework comprises a substantially smooth surface, free of surface alterations. As the magnesium leaches from the magnesium alloy stent framework, after deployment at a target site, the magnesium alloy stent framework surface becomes marred with pores. In another embodiment, the magnesium alloy stent framework received at least one surface modification, such as via mechanical, chemical or electrical means. Mechanical means includes forces such as stamping, machining, EDM wiring or the like, while chemical means includes lithography, plasma argon etching or the like. Creation of surface modifications can increase the surface area of the magnesium alloy stent framework, resulting in a greater amount of magnesium leaching into the body and increased formation of pores, and tissue ingrowth. Any appropriate technique for surface modification can be employed to modify the surface of the magnesium alloy stent framework. Certain mechanical processing techniques may result in undesirable stresses being placed on the stent framework based on the concentration of magnesium within the alloy.

Although the present invention applies to cardiovascular and endovascular stents, the use of magnesium alloyed frameworks may be applied to other implantable and blood-contacting biomedical devices such as coated pacemaker leads, microdelivery pumps, feeding and delivery catheters, heart valves, artificial livers and other artificial organs.

In addition, the magnesium alloy stent framework can be covered with a drug to form a drug eluting stent. The drug can be applied to the bare metal, or the drug can be included within a drug polymer coating, such as disclosed within U.S. patent application Ser. No. 10/674,293, the entirety of which is incorporated herein by reference. Other drug coating techniques can also be used.

While the invention has been described with reference to particular embodiments, it will be understood by one skilled in the art that variations and modifications may be made in form and detail without departing from the spirit and scope of the invention.

What is claimed is:

1. A method for treating a vascular condition, the method comprising:
delivering a magnesium alloy stent framework to a target region of a vessel;
leaching at least a portion of magnesium from the magnesium alloy stent framework; and
forming a plurality of pores within the stent framework of the stent based on the leaching.

2. The method of claim 1 wherein the magnesium alloy stent framework comprises cobalt chromium.

3. The method of claim 1 further comprising:
receiving at least some tissue ingrowth within the formed pores.

4. The method of claim 1 wherein the pores are nanopores.

5. The method of claim 1 wherein the distribution of pores along the length of the stent framework is uncontrolled.

6. The method of claim 1 wherein the distribution of pores along the length of the stent framework is controlled.
